(19) United States
(12) Patent Application Publication
(10) Pub. No.: US 2009/0069659 A1
(43) Pub. Date: Mar. 12, 2009

(54) Blood sugar measurement automatic correction system

(75) Inventor: Daniel C.M. Tseng, Taipei County (TW)

Correspondence Address: Joe McKinney Muncy, PO Box 1364, Fairfax, VA, 22038-1364, US

(73) Assignee: K-JUMP HEALTH CO., LTD.

(21) Appl. No.: 11/898,008

(22) Filed: Sep. 7, 2007

(51) Int. Cl. A61B 5/00

(52) U.S. Cl. 600/365

(57) ABSTRACT

A blood sugar measurement automatic correction system includes a blood sugar measuring unit which has a reaction zone to allow blood and a test agent to generate a chemical reaction to produce electrons, and a test device to measure the produced electrons and output a blood sugar measurement signal to a reading unit, and an error correction unit to receive the blood sugar measurement signal and tune the blood sugar measurement signal according to a correction value set in an error correction zone and output to the reading unit.

### FIELD OF THE INVENTION

The present invention relates to a correction system and particularly to a blood sugar measurement automatic correction system.

### BACKGROUND OF THE INVENTION

To do measurement on a conventional blood sugar measurement equipment has to be done on a medium or large machine. For instance R.O.C. patent publication No. 342447 discloses a polarized optical blood sugar concentration test device which mainly includes (a) a linear polarized dual frequency laser delivering two linear polarized and orthogonal lights of different optical frequencies, (b) a cubical and transparent container without optical activity holding a solution to be tested for optical activity positioned on an optical path behind the dual frequency laser, (c) one set of polarized optical analysis blade located on the optical path behind the cubical container to make the two orthogonal and linear polarized lights to generate a heterodyne interference, (d) an optical detector responsive to fast frequency and a bandpass filter having a center frequency based on a beat frequency of the dual frequencies output from a laser light that are located behind the optical analysis blade to output a heterodyne interference signal having a beat frequency oscillation frequency to be sent to an amplitude measurement equipment, (e) the amplitude of the heterodyne interference signal being received by the amplitude measurement equipment, and (f) a personal computer transforming the amplitude of the heterodyne interference signal to an optical active solution concentration. Nowadays the technology is well developed to shrink the blood sugar measurement equipment to a smaller size usable at home. The home-used blood sugar measurement equipment mainly has a blood sugar test blade coated with a reagent to generate a chemical reaction with blood to release electrons. The electric current intensity resulting from the electrons is used to measure the blood sugar content in the blood.

However, the chemical composition of the reagent is difficult to control accurately in the production process due to external factors such as production environments. Hence the blood contained same amount of blood sugar at different batches of blood sugar test blades could generate different amount of electrons, and affect the test result. There is another approach which inserts a correction blade before measurement or adopts manual correction by entering a correction value before the measurement is done to correct the test result. However, insertion of the correction blade could be inadvertently neglected, or people doing the test could be not familiar with the manual correction process. All this could result in test errors. Hence how to improve the blood sugar test instrument is an issue remained to be resolved in the industry.

### SUMMARY OF THE INVENTION

The primary object of the present invention is to provide a blood sugar measurement automatic correction system that automatically corrects blood sugar measurements through a correction value set in an error connection zone.

To achieve the foregoing object the system according to the invention includes a blood sugar measuring unit which has a reaction zone to allow blood and a test agent to generate a chemical reaction to produce electrons, and a test device to measure the produced electrons and output a blood sugar measurement signal to a reading unit, and an error correction unit to receive the blood sugar measurement signal and tune the blood sugar measurement signal according to a correction value set in an error correction zone and output to the reading unit.

The foregoing, as well as additional objects, features and advantages of the invention will be more readily apparent from the following detailed description, which proceeds with reference to the accompanying drawings.

### BRIEF DESCRIPTION OF THE DRAWINGS

FIG. 1 is a schematic view of the structure of a first embodiment of the invention.

FIG. 2 is a perspective view of the first embodiment of the invention.

FIG. 3 is a fragmentary perspective view of the first embodiment of the invention.

FIGS. 4A, 4B and 4C are schematic views of the structure of a second embodiment of the error correction zone of the invention.

FIGS. 5A, 5B and 5C are schematic views of the structure of a third embodiment of the error correction zone of the invention.

FIGS. 6A, 6B and 6C are schematic views of the structure of a fourth embodiment of the error correction zone of the invention.

FIGS. 7A, 7B and 7C are schematic views of the structure of a fifth embodiment of the error correction zone of the invention.

FIG. 8 is a schematic view of the structure of a sixth embodiment of the error correction zone of the invention.

FIGS. 9A and 9B are schematic views of the structure of a seventh embodiment of the error correction zone of the invention.

### DETAILED DESCRIPTION OF THE PREFERRED EMBODIMENTS

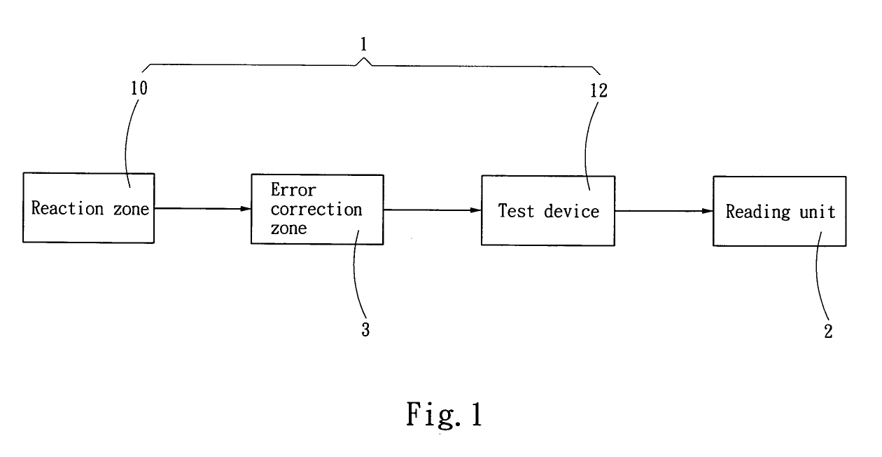

Please refer to FIG. 1 for the structure of a first embodiment of the invention. The invention provides a blood sugar measurement automatic correction system. It includes:

a blood sugar measuring unit 1 which has a reaction zone 10 to generate a chemical reaction between blood and a test agent (such as enzyme oxide) to produce electrons and a test device 12 to measure the electrons produced at the reaction zone 10 to output a blood sugar measurement signal to a reading unit 2. The reading unit 2 may include a display device, a sound output device or a voice output device (not shown in the drawings) to inform users the blood sugar measurement value in a form of images, sound or voice; and

an error correction unit (not shown in the drawings) which receives the blood sugar measurement signal and tunes the blood sugar measurement value according to a correction value set by an error correction zone 3 (to be discussed later), and outputs to the reading unit 2. In this embodiment the error correction unit is located in the reading unit 2. When the blood sugar measuring unit 1 is inserted into the reading unit 2, blood is dripped to the reaction zone 10 to allow a chemical reaction to complete within a selected time period (such as 5 to 7 seconds) between the blood and the test agent. The test device 12 outputs the blood sugar measurement signal to the reading unit 2. By in cooperating with the correction value of the error correction zone 3 obtained by the error correction unit, a test result of the blood sugar measurement signal is tuned to form a correct blood sugar measurement value to inform the users through the display device, sound output device or voice output device.

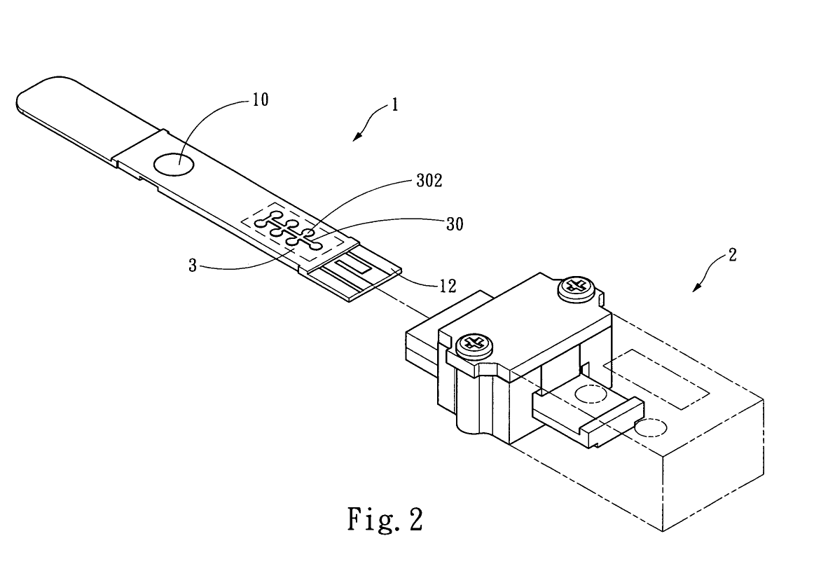

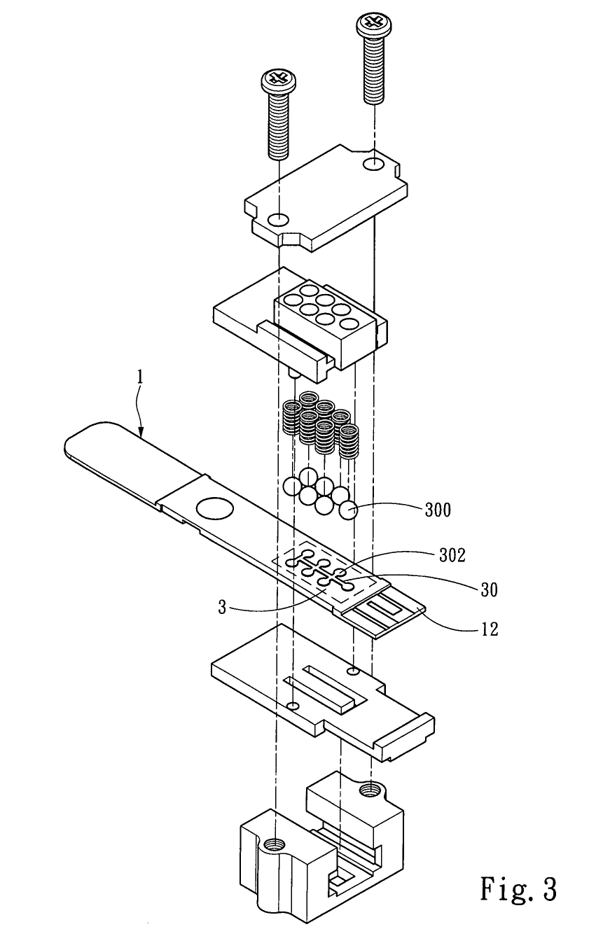

Refer to FIGS. 2 and 3 for a first embodiment of the invention. The error correction zone 3 includes a circuit portion 30 which determines electric power conductive amount through circuit ON and OFF conditions caused by whether beads 300 are held on apertures 302. Through the electric power conductive amount the correction value is set, thereby the reading unit 2 can detect the measurement result and tune to form a base value.

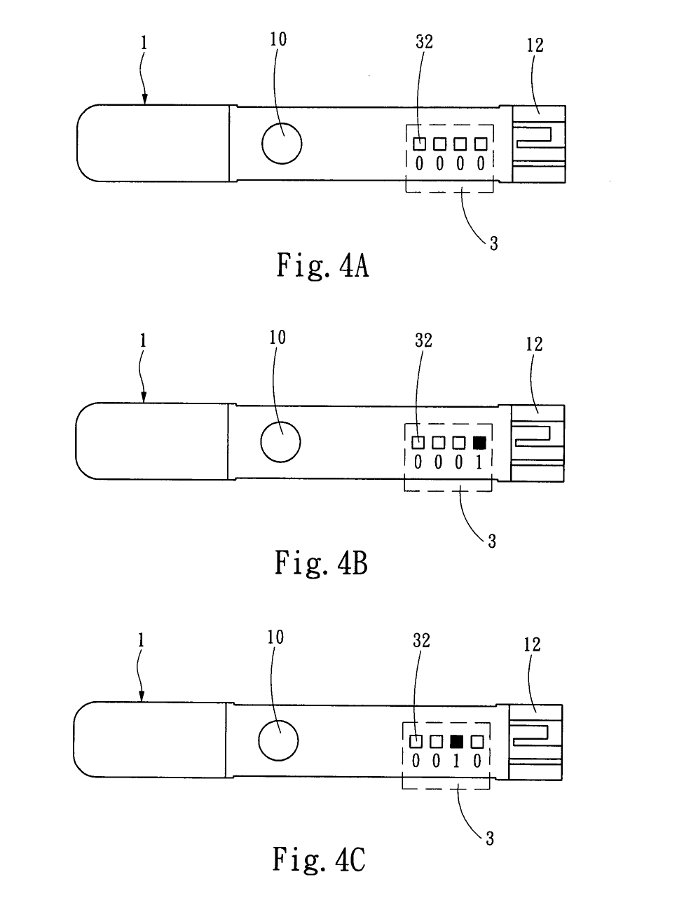

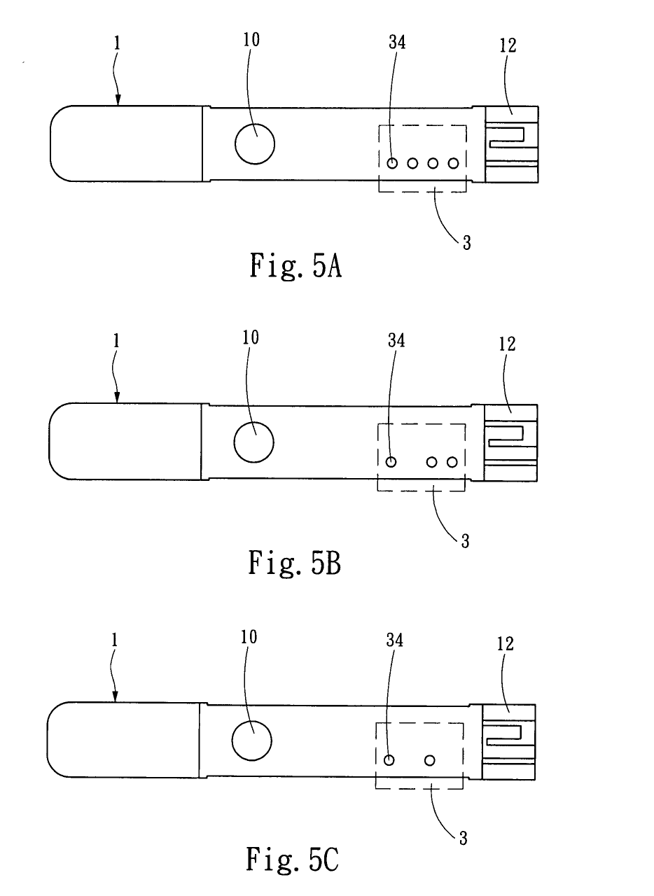

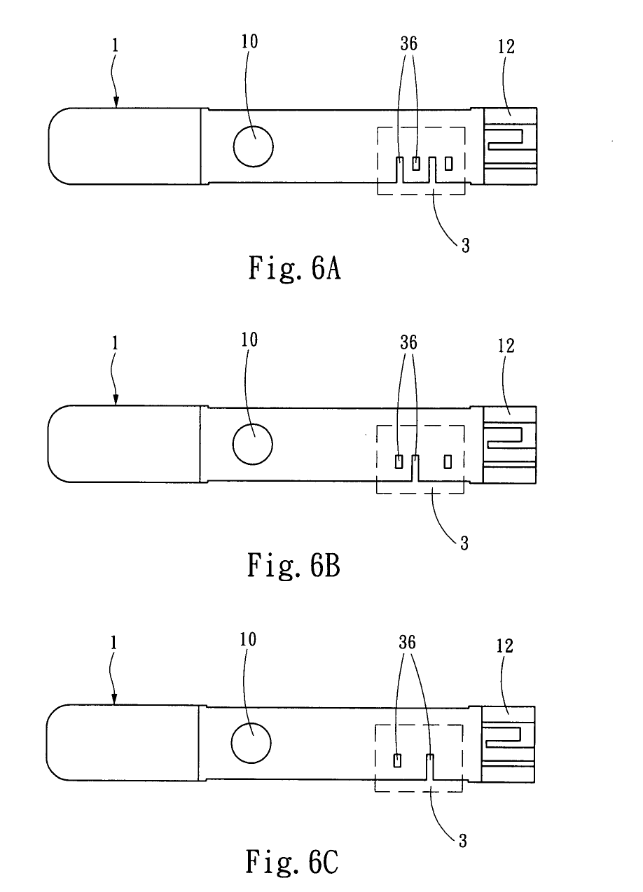

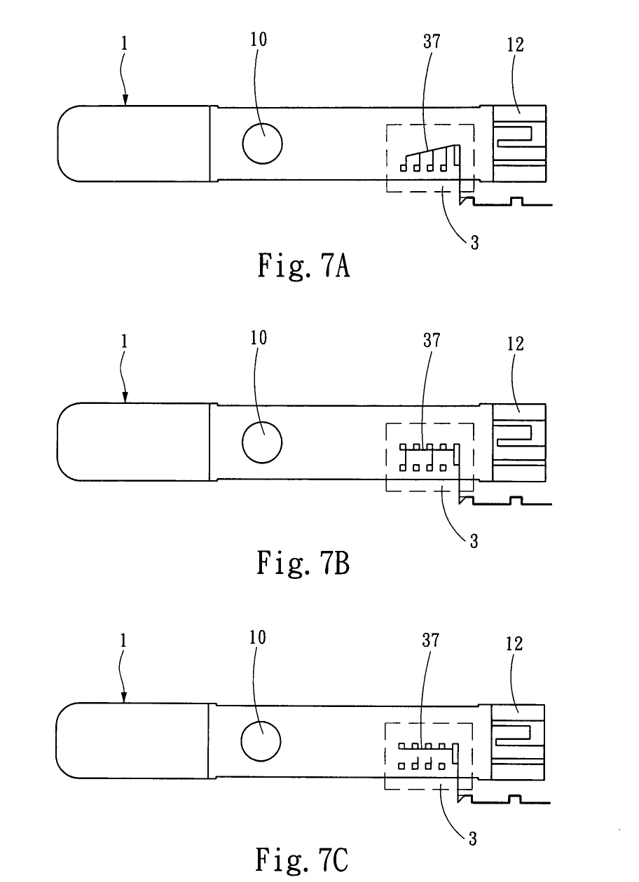

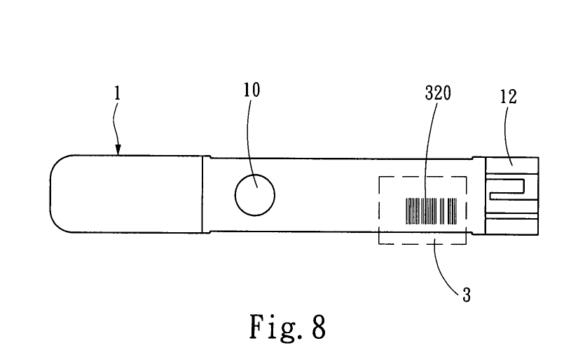

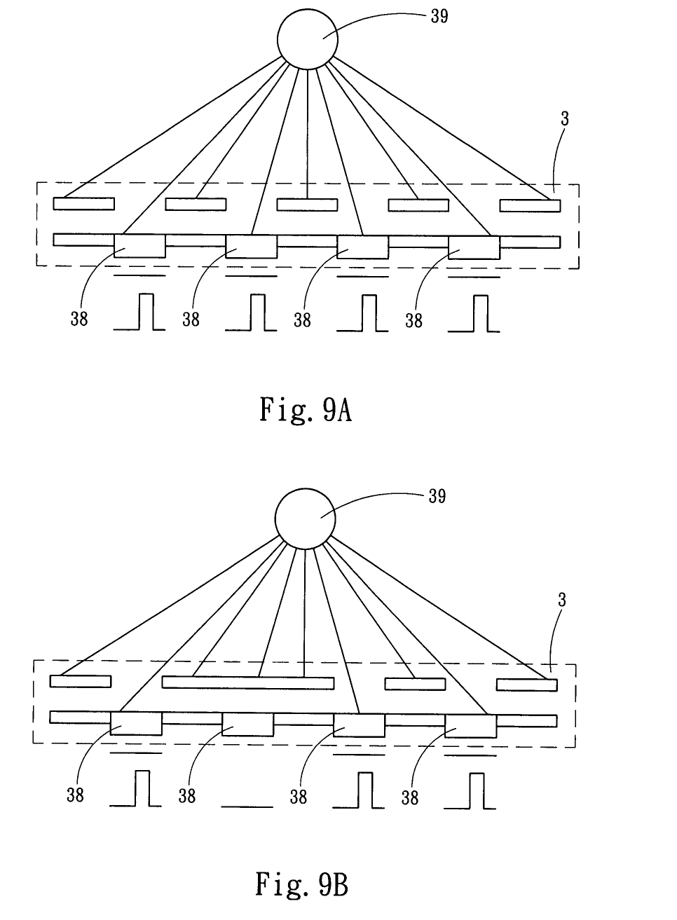

Refer to FIGS. 4A, 4B and 4C for a second embodiment of the invention. The error correction zone 3 has a color segment 32 to set the correction value through the color of a color patch located thereon. FIGS. 5A, 5B and 5C illustrate a third embodiment of the invention. The error correction zone 3 has a punch portion 34 to set the correction value through the distribution of punched holes. FIGS. 6A, 6B and 6C illustrate a fourth embodiment of the invention. The correction zone 3 has a teeth portion 36 to set the correction value through the jagged condition thereof. FIGS. 7A, 7B and 7C illustrate a fifth embodiment of the invention. The correction zone 3 has a metal connective portion 37 to set the correction value through an electric conductive condition thereof. FIG. 8 illustrates a sixth embodiment of the invention. The error correction zone 3 has an encoding portion 320 to set the correction value through a data code (such as an optical barcode). FIGS. 9A and 9B illustrate a seventh embodiment of the invention. The error correction zone 3 has an optical response portion 38 which receives light projected from a light source 30 to form a transparent condition to set the correction value.

As a conclusion, the invention automatically corrects the measured result of each blood sugar measurement through the error correction zone 3. Thus the measurement errors of the conventional techniques resulting from user's forgetting or unfamiliar to do correction can be prevented. It is a significant improvement over the conventional techniques.

While the preferred embodiments of the invention have been set forth for the purpose of disclosure, modifications of the disclosed embodiments of the invention as well as other embodiments thereof may occur to those skilled in the art. Accordingly, the appended claims are intended to cover all embodiments which do not depart from the spirit and scope of the invention.

What is claimed is:

1. A blood sugar measurement automatic correction system, comprising:
a blood sugar measuring unit which has a reaction zone to generate a chemical reaction between blood and a test agent to produce electrons and a test device to measure the electrons produced at the reaction zone to output a blood sugar measurement signal to a reading unit; and
an error correction unit which receives the blood sugar measurement signal and tunes the blood sugar measurement value according to a correction value set by an error correction zone, and outputs to the reading unit.

2. The blood sugar measurement automatic correction system of claim 1, wherein the error correction zone includes a color segment to set the correction value through the color of a color patch located thereon.

3. The blood sugar measurement automatic correction system of claim 1, wherein the error correction zone includes a metal connective portion to set the correction value through an electric conductive condition.

4. The blood sugar measurement automatic correction system of claim 1, wherein the error correction zone includes an optical response portion to set the correction value through a transparent condition.

5. The blood sugar measurement automatic correction system of claim 1, wherein the error correction zone includes a teeth portion to set the correction value through a jagged condition.

6. The blood sugar measurement automatic correction system of claim 1, wherein the error correction zone includes an encoding portion to set the correction value through a data code’

7. The blood sugar measurement automatic correction system of claim 1, wherein the error correction zone includes a punch portion to set the correction value through distribution of punched holes.

8. The blood sugar measurement automatic correction system of claim 1, wherein the error correction zone includes a circuit portion to set the correction value through circuit ON and OFF conditions.

9. The blood sugar measurement automatic correction system of claim 1, wherein the test agent is an enzyme oxide.

10. The blood sugar measurement automatic correction system of claim 1, wherein the reading unit is a display device to display a blood sugar measurement value contained in the blood sugar measurement signal.

11. The blood sugar measurement automatic correction system of claim 1, wherein the reading unit has a voice output device to broadcast a blood sugar measurement value contained in the blood sugar measurement signal.

12. The blood sugar measurement automatic correction system of claim 1, wherein the reading unit has a sound output device to output sound for a blood sugar measurement value contained in the blood sugar measurement signal.
